(19) United States
(12) Patent Application Publication
(10) Pub. No.: US 2025/0359797 A1
(43) Pub. Date: Nov. 27, 2025

(54) ELECTROCARDIOGRAM-PHONOCARDIOGRAM SYNCHRONOUS ACQUISITION DEVICE AND ELECTROCARDIOGRAM-PHONOCARDIOGRAM DETECTION SYSTEM

(71) Applicant: Beijing University of Technology, Beijing (CN)

(72) Inventors: Guangyu Bin, Beijing (CN); Meitong Zhu, Beijing (CN)

(21) Appl. No.: 19/213,000

(22) Filed: May 20, 2025

(30) Foreign Application Priority Data
May 21, 2024 (CN) 2024106339130

(51) Int. Cl. A61B 5/28; A61B 5/00; A61B 5/252; A61B 7/02

(52) U.S. Cl. CPC A61B 5/28; A61B 5/252; A61B 5/6834; A61B 7/02; A61B 2560/0468; A61B 2562/227

(57) ABSTRACT

An electrocardiogram (ECG)-phonocardiogram (PCG) synchronous acquisition device and an ECG-PCG detection system are provided. The ECG-PCG synchronous acquisition device includes a conductive adsorption unit configured to be absorbed to human skin; and an acquisition unit connected to the conductive adsorption unit. The acquisition unit is configured to acquire ECG signals directly, and simultaneously acquire PCG signals through the conductive adsorption unit. The ECG-PCG synchronous acquisition device and the ECG-PCG detection system are used to solve the technical problem of low efficiency in synchronously acquiring electrocardiogram signals and heart sound signals in the related art.

### CROSS-REFERENCE TO RELATED APPLICATION

This application claims priority to Chinese Patent Application No. 202410633913.0, filed May 21, 2024, which is herein incorporated by reference in its entirety.

### TECHNICAL FIELD

The disclosure relates to the technical field of medical devices, and more particularly to an electrocardiogram (ECG) and phonocardiogram (PCG) synchronous acquisition device and an ECG-PCG detection system.

### BACKGROUND

ECG and PCG are two conventional means of detecting cardiovascular diseases. ECG detection typically uses limb electrode clips and suction cup electrodes, where the suction cup electrodes are fixed to the chest by negative pressure to acquire ECG signals. For PCG detection, doctors usually manually press the stethoscope against specific positions on the chest to acquire PCG signals (also referred to as heart sound signals). Synchronous acquisition of ECG and PCG signals can provide complementary information, reveal key cardiovascular parameters that cannot be obtained from either signal alone, and has significant clinical value.

In the related art, when it is necessary to synchronously acquire the ECG and PCG signals, the suction cup electrodes should be attached to the chest to acquire the ECG signals first, and then a PCG sensor should be manually pressed and held in a specific position on the chest to acquire the PCG signals, affecting the efficiency of signal acquisition.

### SUMMARY

The disclosure provides an ECG-PCG synchronous acquisition device and an ECG-PCG detection system, to solve technical problems of low efficiency in synchronously acquiring ECG signals and PCG signals in the related art.

The disclosure provides the ECG-PCG synchronous acquisition device, including a conductive adsorption unit and an acquisition unit. The conductive adsorption unit is configured to be absorbed onto human skin. The acquisition unit, connected to the conductive adsorption unit, is configured to acquire ECG signals directly and simultaneously obtain PCG signals through the conductive adsorption unit.

In an embodiment, the conductive adsorption unit includes: a conductive assembly, a hollow suction ball and a connecting tube. The conductive assembly defines an inner cavity, an adsorption port and a connecting opening, and the adsorption port and the connecting opening are connected to the inner cavity. The hollow suction ball defines a negative pressure cavity and an air intake port connected to the negative pressure cavity. An end of the connecting tube is disposed at the connecting opening, and another end of the connecting tube is disposed at the air intake port.

In an embodiment, the conductive assembly includes an adsorption port, and a circular connecting end face positioned diametrically opposite to each other. The adsorption port is defined on a bottom of the conductive adsorption unit. A diameter of the inner cavity tapers along a direction from the adsorption port to the circular connecting end face.

In an embodiment, the circular connecting end face defines a first placement interface, the acquisition unit is mounted over the first placement interface, and the connecting opening is defined on a sidewall of the conductive assembly.

In an embodiment, a sidewall of the connecting tube defines an insertion opening. The acquisition unit is slidably movable within the connecting tube and in sealing fit with the air intake port. When the adsorption port is adsorbed to the human skin, the acquisition unit extends into the connecting tube to separate the inner cavity from the negative pressure cavity.

In an embodiment, a partition part is disposed in the inner cavity and configured to divide the inner cavity into an adsorption cavity and a resonance cavity. The adsorption cavity is disposed surrounding the resonance cavity, and the resonance cavity is configured to conduct the PCG signals to the acquisition unit. The partition part (resonance cavity) includes a first end and a second end disposed sequentially in that order along the direction from the adsorption port to the circular connecting end face. The first and second ends define the adsorption port and a second placement interface respectively. The acquisition unit is entirely disposed in the adsorption cavity, an end of the acquisition unit is mounted over the second placement interface, another end of the acquisition unit extends through a sealed enclosure (i.e., an enclosure of the adsorption cavity). The negative pressure cavity and the adsorption cavity are fluidically connected via the adsorption cavity's circular end face, ensuring unimpeded airflow between them.

In an embodiment, the first end (adsorption port) defines an opening, a diaphragm is disposed at the opening, and when the adsorption port is adsorbed onto the human skin, the diaphragm is in contact with the human skin.

In an embodiment, a diameter of the resonance cavity tapers along the direction from the adsorption port to the circular connecting end face.

In an embodiment, the conductive assembly is provided with a lead part, and the lead part is a first cable configured to be connected to an ECG machine.

The disclosure further provides the ECG-PCG detection system, including: an ECG machine, and the ECG-PCG synchronous acquisition device connected to the ECG machine through a second cable.

The ECG-PCG synchronous acquisition device and the ECG-PCG detection system of the disclosure has following characteristics and advantages.

The ECG-PCG synchronous acquisition device of the disclosure achieves ECG-PCG synchronous acquisition by integrating the conductive adsorption unit with the acquisition unit. The design of the ECG-PCG synchronous acquisition device overcomes problems of complicated operation and low efficiency associated with traditional methods that require separate acquisition of the ECG and PCG signals. The conductive adsorption unit not only ensures stable adhesion to the human skin to guarantee continuous ECG signal acquisition but also serves as a bridge that enables the acquisition unit on the device to simultaneously obtain the PCG signals, eliminating the need for additional manual positioning and continuous pressing, and significantly enhancing the convenience and efficiency of the synchronous acquisition. This synchronous acquisition method simplifies the medical detection process and reduces operation time.

### BRIEF DESCRIPTION OF DRAWINGS

In order to more clearly illustrate technical solutions of the disclosure or in the related art, a brief introduction will be given to the accompanying drawings required for description of embodiments or the related art. It is apparent that the accompanying drawings described below are some of the embodiments of the disclosure. For those skilled in the art, other drawings can be obtained based on these drawings without creative labor.

FIG. 1 illustrates a front view of an ECG-PCG synchronous acquisition device according to a first embodiment of the disclosure.

FIG. 2 illustrates a sectional view of the ECG-PCG synchronous acquisition device illustrated in FIG. 1.

FIG. 3 illustrates a front view of an ECG-PCG synchronous acquisition device according to a second embodiment of the disclosure.

FIG. 4 illustrates a sectional view of the ECG-PCG synchronous acquisition device illustrated in FIG. 3.

FIG. 5 illustrates a front view of an ECG-PCG synchronous acquisition device according to a third embodiment of the disclosure.

FIG. 6 illustrates a sectional view of the ECG-PCG synchronous acquisition device illustrated in FIG. 5.

Description of reference signs: 100: conductive adsorption unit; 110: conductive assembly; 111: inner cavity; 1101: adsorption cavity; 1102: resonance cavity; 112: adsorption port; 113: connecting opening; 115: circular connecting end face; 1151: first placement interface; 116: partition part; 1161: first end; 1162: second end; 1163: diaphragm; 1164: second placement interface; 1165: opening; 117: circular end face; 120: hollow suction ball; 121: negative pressure cavity; 122: air intake port; 130: connecting tube; 131: insertion opening; 140: lead part; 200: acquisition unit; 300: second cable.

### DETAILED DESCRIPTION OF EMBODIMENTS

In order to clarify the purposes, technical solutions, and advantages of the disclosure, a clear and complete description of the technical solutions of the disclosure is provided below in conjunction with the accompanying drawings of the disclosure. Apparently, the illustrated embodiments are some of the embodiments of the disclosure, not all of them. Based on the embodiments of the disclosure, all other embodiments obtained by those skilled in the art without creative labor are within the scope of protection of the disclosure.

In the description of the embodiments, it should be understood that the terms “center”, “longitudinal”, “transverse”, “length”, “width”, “thickness”, “up”, “below”, “front”, “back”, “left”, “right”, “vertical”, “horizontal”, “top”, “bottom”, “inside”, “outside”, “clockwise”, “counterclockwise”, “axial”, “radial”, “circumferential” and other directional or positional relationships indicated are based on the directional or positional relationships shown in the accompanying drawings, are only for the convenience of describing the embodiments and simplifying the description, and do not indicate or imply the device or device referred to must have a specific orientation, be constructed and operated in a specific orientation, and therefore cannot be understood as a limitation on the embodiments.

In addition, terms “first” and “second” are only used for descriptive purposes and cannot be understood as indicating or implying relative importance or implying the number of technical features indicated. Therefore, the features that are limited to “first” and “second” can explicitly or implicitly include at least one of these features. In the description of the embodiments, the meaning of “multiple” is at least two, such as two and three, etc., unless otherwise specified.

In the embodiments, unless otherwise specified and limited, terms “setting”, “installation”, “connection”, and “fixation” etc., should be broadly understood, for example, it can be a fixed connection, a detachable connection, or integrated; it can be a mechanical connection or an electrical connection; it can be directly connected or indirectly connected through an intermediate medium, and can be a connection within two components or an interaction relationship between two components, unless otherwise specified. For those skilled in the art, specific meanings of the above terms in the embodiments can be understood according to the specific situation.

In the embodiments of the disclosure, unless otherwise specified and limited, when the first feature is “on” or “under” the second feature, the first feature may be in direct contact with the second feature, or the first and second features may be in indirect contact through an intermediate medium. Moreover, the first feature being “on” or “above” the second feature may indicate that the first feature is directly above or diagonally above the second feature, or simply that the first feature is horizontally higher than the second feature. The first feature being “under” or “below” the second feature may indicate that the first feature can be located directly or diagonally below the second feature, or simply indicate that the first feature has a lower horizontal height than the second feature.

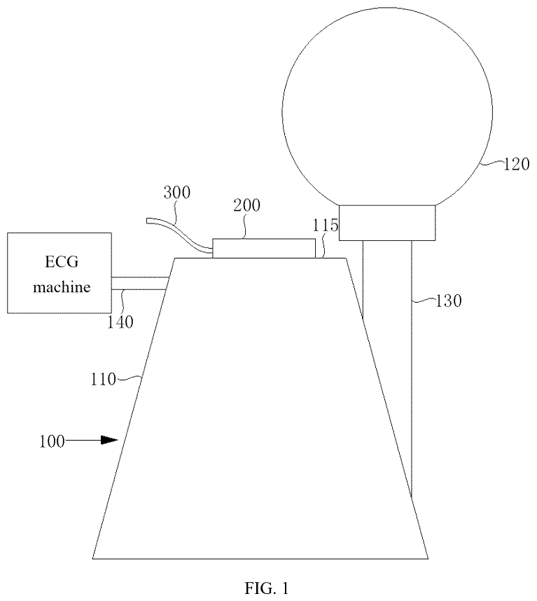

FIGS. 1-6 illustrate an ECG-PCG synchronous acquisition device provided by the disclosure. As shown in figures, the disclosure provides the ECG-PCG synchronous acquisition device, and the ECG-PCG synchronous acquisition device includes: a conductive adsorption unit 100 and an acquisition unit 200. The conductive adsorption unit 100 is configured to be absorbed onto human skin. The acquisition unit 200 is connected to the conductive adsorption unit 100. The acquisition unit 200 is configured to acquire ECG signals directly, and simultaneously acquire PCG signals through the conductive adsorption unit 100.

The ECG-PCG synchronous acquisition device of the disclosure achieves ECG-PCG synchronous acquisition by integrating the conductive adsorption unit 100 with the acquisition unit 200. The design of the ECG-PCG synchronous acquisition device overcomes the problems of complicated operation and low efficiency associated with traditional methods that require separate acquisition of the ECG and PCG signals. The conductive adsorption unit 100 not only ensures stable adhesion to the human skin to guarantee continuous ECG signal acquisition but also serves as a bridge that enables the acquisition unit 200 on the device to simultaneously obtain the PCG signals, eliminating the need for additional manual positioning and continuous pressing, and significantly enhancing the convenience and efficiency of the synchronous acquisition. This synchronous acquisition method simplifies the medical detection process and reduces operation time.

In the embodiment, the acquisition unit 200 may be an acquisition board provided with a PCG sensor (i.e., a heart sound sensor). The PCG sensor is configured to detect the PCG signals. The conductive adsorption unit 100 may be a structural component made of a conductive metal material. The acquisition board may be electrically connected to the conductive adsorption unit 100. The conductive adsorption unit 100 can be in contact with skin to conduct ECG signals to the acquisition board.

In an embodiment, the conductive adsorption unit 100 includes: a conductive assembly 110, a hollow suction ball 120 and a connecting tube 130. The conductive assembly 110 defines an inner cavity 111, an adsorption port 112 and a connecting opening 113, and the adsorption port 112 and the connecting opening 113 are connected to the inner cavity 111. The hollow suction ball 120 defines a negative pressure cavity 121 and an air intake port 122, and the air intake port 122 is connected to the negative pressure cavity 121. An end of the connecting tube 130 is disposed at the connecting opening 113, and another end of the connecting tube 130 is disposed at the air intake port 122.

Specifically, the conductive component 110 serves the function of electrical conductivity, and the structure of the inner cavity 111 and the design of the connecting opening 113 ingeniously make the inner cavity 111 and the connecting opening 113 connected to the negative pressure cavity 121 and the air intake port 122 of the hollow suction ball 120, allowing the adsorption port 112 to adhere to the skin. For example, during acquisition, the hollow suction ball 120 can be pressed first, and then the adsorption port 112 is attached against the human skin to ensure good contact between the edge of the adsorption port 112 and the skin, thereby enhancing the quality of ECG signal acquisition. In this way, the structural design not only enhances the stability and comfort of device usage, but also, through the ingenious combination of physical mechanisms, effectively avoids signal interference problems caused by movement or unstable pressing in traditional methods, thus ensuring the high efficiency and high quality of synchronous ECG and PCG signal acquisition, and further improving the reliability of clinical detection and the patient experience.

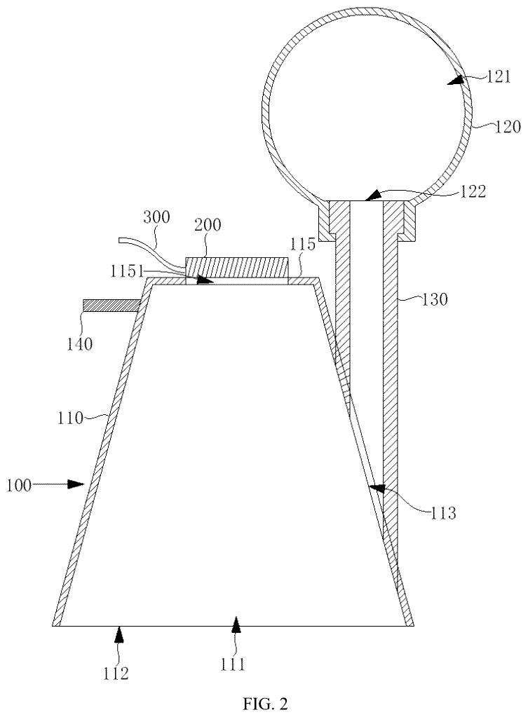

In a first embodiment, as shown in FIGS. 1-2, the conductive assembly 110 includes an adsorption port 112 and a circular connecting end face 115 disposed diametrically opposite to each other. The adsorption port 112 is defined on a bottom of the conductive adsorption unit 100. A diameter of the inner cavity 111 tapers along a direction from the adsorption port 112 to the circular connecting end face 115.

In the first embodiment, the circular connecting end face 115 defines a first placement interface 1151, the acquisition unit 200 is mounted over the first placement interface 1151, and the connecting opening 113 is defined on a sidewall of the conductive assembly 110.

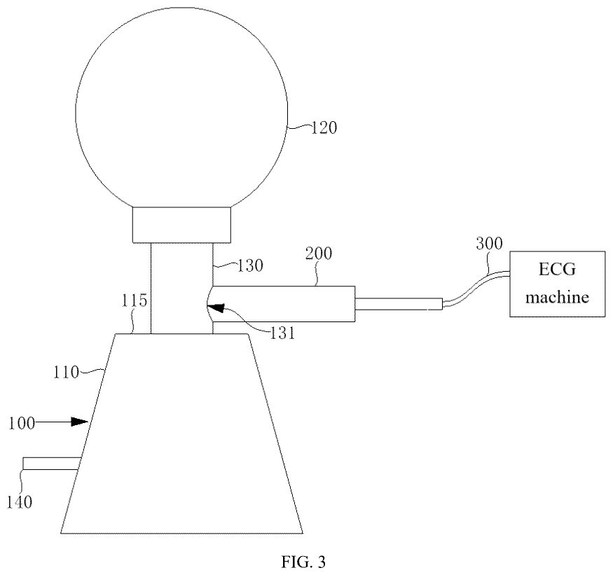

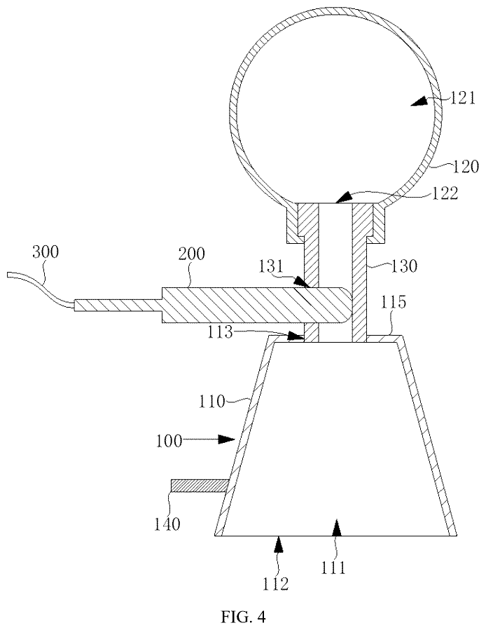

In a second embodiment, as shown in FIGS. 3-4, a sidewall of the connecting tube 130 defines an insertion opening 131. The acquisition unit 200 is slidably movable within the connecting tube, and in sealing fit with the air intake port 122. When the adsorption port 112 is adsorbed onto the human skin, the acquisition unit 200 can extend into the connecting tube 130 to separate the inner cavity 111 from the negative pressure cavity 121.

Specifically, since the internal passage of the connecting tube 130 and the negative pressure cavity 121 of the hollow suction ball 120 may affect the acquisition of PCG signals, by setting the above structures, the acquisition unit 200 is disposed outside the connecting tube 130 under normal conditions; and when acquisition is required, the hollow suction ball 120 can be squeezed first to be in a compressed state, and then, the adsorption port 112 is placed against the human skin, followed by releasing the hollow suction ball 120. At this point, the hollow suction ball 120 generates a negative pressure, making the adsorption port 112, acting like a suction cup/disc, adhere to the human skin under the effect of the negative pressure. After confirming that the adsorption port 112 is securely adsorbed to the skin, the acquisition unit 200 can be manually pushed to extend into the connecting tube 130, thereby separating the inner cavity 111 and the negative pressure cavity 121.

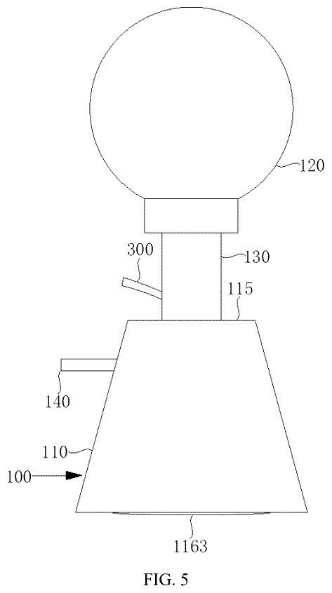

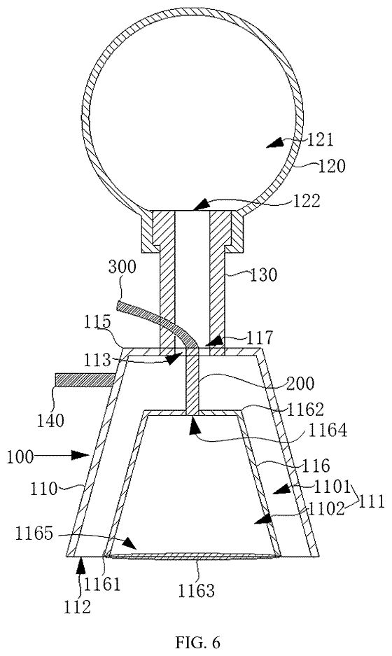

In a third embodiment, as shown in FIGS. 5-6, a partition part 116 is disposed in the inner cavity 111, for example, the partition part 116 is disposed in the inner cavity 111 through a connecting piece, and the partition part 116 can divide the inner cavity 111 into an adsorption cavity 1101 and a resonance cavity 1102. The adsorption cavity 1101 is disposed surrounding the resonance cavity 1102, and the resonance cavity 1102 is configured to conduct the PCG signals to the acquisition unit 200. The partition part 116 includes a first end 1161 and a second end 1162 disposed sequentially in that order along the direction from the adsorption port 112 to the circular connecting end face 115. The first end 1161 can be in contact with skin. The first end 1161 and the second end 1162 define the adsorption port 112 and a second placement interface 1164 respectively. The acquisition unit 200 is entirely disposed in the adsorption cavity 1101, an end of the acquisition unit 200 is mounted over the second placement interface 1164, another end of the acquisition unit 200extends through an enclosure of the adsorption cavity 1101, and the negative pressure cavity 121 and the adsorption cavity 1101 are fluidically connected via a circular end face (i.e., a gap) 117 of the adsorption cavity 1101 to ensure unimpeded airflow therebetween.

Specifically, by setting the partition part 116 in the inner cavity 111, the inner cavity 111 is ingeniously divided into the adsorption cavity 1101 and the resonance cavity 1102. The layout where the adsorption cavity 1101 surrounds the resonance cavity 1102 ensures not only good adhesion of the suction ball to the skin but also provides a dedicated pathway for the conduction of PCG signals. The resonance cavity 1102 focuses on acquiring and conducting the PCG signals to the acquisition unit 200. Through precise acoustic design, the clarity and signal-to-noise ratio of the PCG signals can be effectively enhanced, making analysis more accurate. The design of the two ends of the partition part 116, especially the second placement interface 1164 at the second end 1162, allows a part of the acquisition unit 200 to pass through in a sealed manner and extend to the vicinity of the connecting opening 113, forming the tiny gap 117 for air circulation. This ingenious arrangement not only ensures the effective conduction of PCG signals but also guarantees the realization of the adsorption function. At the same time, this structure creates a relatively independent and optimized environment for the acquisition of PCG signals, reducing the risk of cross-interference, enhancing the overall signal quality and reliability of analysis, and further consolidating the superior performance of the ECG-PCG synchronous acquisition device in clinical detection and long-term monitoring.

In an embodiment, the first end 1161 defines an opening 1165, a diaphragm 1163 is disposed at the opening 1165, and when the adsorption port 112 is adsorbed to the human skin, the diaphragm 1163 is in contact with the human skin.

Specifically, when the adsorption port 112 is adhered to the human skin, the diaphragm 1163 is in close contact with the skin. Mimicking the principle of a stethoscope, the PCG signals are efficiently received through the vibration of the diaphragm 1163, thereby further enhancing the sensitivity and accuracy of PCG signal acquisition.

In this embodiment, a diameter of the resonance cavity 1102 tapers along the direction from the adsorption port 112 to the circular connecting end face 115.

In an embodiment, the conductive assembly 110 is provided with a lead part 140, and the lead part 140 is a first cable configured to be connected to an ECG machine.

Specifically, when only the ECG signals need to be acquired, there is no need to connect the acquisition unit 200 with the ECG machine, and the ECG machine can be connected to the conductive adsorption unit 100 through the first cable.

The disclosure further provides an ECG-PCG detection system. The ECG-PCG detection system includes an ECG machine and the ECG-PCG synchronous acquisition device. The ECG-PCG synchronous acquisition device (the acquisition unit 200) is connected to the ECG machine through a second cable 300.

Specifically, the ECG-PCG synchronous acquisition device of the ECG-PCG detection system has specific structures, working principles, and beneficial effects same as those of the above embodiments, which are not repeated here.

In the embodiment, the second cable 300 may include four wires consisting of a power supply wire, a ground wire, an ECG signal wire and a PCG signal wire.

In the description herein, references to terms “an embodiment”, “some embodiments”, “method”, “specific method”, or “some methods” mean that the specific features, structures, materials, or characteristics described in conjunction with the embodiments or implementations are included in at least one embodiment or implementations of the disclosure. The schematic expressions of the above terms herein do not necessarily refer to the same embodiments or implementations. Moreover, the specific features, structures, materials, or characteristics described can be combined in any one or more embodiments or implementations in an appropriate manner. In addition, without contradiction, those skilled in the art can combine and integrate the different embodiments or implementations described in this specification, as well as the features of different embodiments or implementations.

Finally, it should be noted that the above embodiments are only used to illustrate and not to limit the technical solutions of the disclosure; Although the disclosure has been described in detail with reference to the aforementioned embodiments, those skilled in the art should understand that they can still modify the technical solutions described in the aforementioned embodiments, or equivalently replace some of the technical features; And these modifications or substitutions do not depart from the essence and scope of the corresponding technical solutions of the embodiments of the disclosure.

What is claimed is:

1. An electrocardiogram (ECG)-phonocardiogram (PCG) synchronous acquisition device, comprising:
a conductive adsorption unit (100), configured to be absorbed onto human skin; and
an acquisition unit (200), connected to the conductive adsorption unit (100), wherein the acquisition unit (200) is configured to acquire ECG signals directly, and simultaneously acquire PCG signals through the conductive adsorption unit (100).

2. The ECG-PCG synchronous acquisition device as claimed in claim 1, wherein the conductive adsorption unit (100) comprises: a conductive assembly (110), a hollow suction ball (120) and a connecting tube (130);
wherein the conductive assembly (110) defines an inner cavity (111), an adsorption port (112) and a connecting opening (113), and the adsorption port (112) and the connecting opening (113) are connected to the inner cavity (111);
wherein the hollow suction ball (120) defines a negative pressure cavity (121) and an air intake port (122) connected to the negative pressure cavity (121); and
wherein an end of the connecting tube (130) is disposed at the connecting opening (113), and another end of the connecting tube (130) is disposed at the air intake port (122).

3. The ECG-PCG synchronous acquisition device as claimed in claim 2, wherein the conductive assembly (110) comprises an adsorption port (112) and a circular connecting end face (115) positioned diametrically opposite to each other;
wherein the adsorption port (112) is defined on a bottom of the conductive adsorption unit (100); and
wherein a diameter of the inner cavity (111) tapers along a direction from the adsorption port (112) to the circular connecting end face (115).

4. The ECG-PCG synchronous acquisition device as claimed in claim 3, wherein the circular connecting end face (115) defines a first placement interface (1151), the acquisition unit (200) is mounted over the first placement interface (1151), and the connecting opening (113) is defined on a sidewall of the conductive assembly (110).

5. The ECG-PCG synchronous acquisition device as claimed in claim 3, wherein a sidewall of the connecting tube (130) defines an insertion opening (131);
wherein the acquisition unit (200) is slidably movable within the connecting tube (130) and in sealing fit with the air intake port (122); and
wherein when the adsorption port (112) is adsorbed to the human skin, the acquisition unit (200) extends into the connecting tube (130) to separate the inner cavity (111) from the negative pressure cavity (121).

6. The ECG-PCG synchronous acquisition device as claimed in claim 3, wherein a partition part (116) is disposed in the inner cavity (111) and configured to divide the inner cavity (111) into an adsorption cavity (1101) and a resonance cavity (1102), the adsorption cavity (1101) is disposed surrounding the resonance cavity (1102), and the resonance cavity (1102) is configured to conduct the PCG signals to the acquisition unit (200);
wherein the partition part (116) comprises a first end (1161) and a second end (1162) disposed sequentially in that order along the direction from the adsorption port (112) to the circular connecting end face (115);
wherein the first end (1161) and the second end (1162) define the adsorption port (112) and a second placement interface (1164) respectively; and
wherein the acquisition unit (200) is entirely disposed in the adsorption cavity (1101), an end of the acquisition unit (200) is mounted over the second placement interface (1164), another end of the acquisition unit (200) extends through an enclosure of the adsorption cavity (1101), and the negative pressure cavity (121) and the adsorption cavity (1101) are fluidically connected via a circular end face (117) of the adsorption cavity (1101) to ensure unimpeded airflow therebetween.

7. The ECG-PCG synchronous acquisition device as claimed in claim 6, wherein the first end (1161) defines an opening (1165), a diaphragm (1163) is disposed at the opening (1165), and when the adsorption port (112) is adsorbed onto the human skin, the diaphragm (1163) is in contact with the human skin.

8. The ECG-PCG synchronous acquisition device as claimed in claim 6, wherein a diameter of the resonance cavity (1102) tapers along the direction from the adsorption port (112) to the circular connecting end face (115).

9. The ECG-PCG synchronous acquisition device as claimed in claim 1, wherein the conductive assembly (110) is provided with a lead part (140), and the lead part (140) is a first cable configured to be connected to an ECG machine.

10. An ECG-PCG detection system, comprising:
an ECG machine; and
an ECG-PCG synchronous acquisition device as claimed in claim 1, connected to the ECG machine through a second cable (300).
